(19) United States
(12) Patent Application Publication
(10) Pub. No.: US 2024/0070885 A1
(43) Pub. Date: Feb. 29, 2024

(54) SKELETON ESTIMATING METHOD, DEVICE, NON-TRANSITORY COMPUTER-READABLE RECORDING MEDIUM STORING PROGRAM, SYSTEM, TRAINED MODEL GENERATING METHOD, AND TRAINED MODEL

(71) Applicant: SHISEIDO COMPANY, LTD., Tokyo (JP)

(72) Inventor: Noriko HASEGAWA, Tokyo (JP)

(21) Appl. No.: 18/261,508

(22) PCT Filed: Feb. 15, 2022
(86) PCT No.: PCT/JP2022/005908

§ 371 Date: Jul. 14, 2023

(30) Foreign Application Priority Data
Feb. 15, 2021 (JP) 2021-021915

(51) Int. Cl. G06T 7/50; G06V 10/774; G06V 40/16

(52) U.S. Cl. CPC G06T 7/50; G06V 10/774; G06V 40/165; G06V 40/171; G06T 2207/20081; G06T 2207/30201

(57) ABSTRACT

To obtain a shape relating to a facial skeleton easily, a method according to an embodiment of the present invention includes a step of determining a nose feature of a user and a step of estimating a shape relating to a facial skeleton of the user based on the nose feature of the user.

### TECHNICAL FIELD

The present invention relates to a skeleton estimating method, a device, a program, a system, a trained model generating method, and a trained model.

### BACKGROUND OF THE INVENTION

Hitherto, three-dimensional facial features have been utilized in the fields of, for example, beauty care (Patent Document 1). Examples of three-dimensional facial features include the shape of the facial skeleton itself, and the shape of the face attributable to the skeleton (hereinafter, referred to as “a shape relating to a facial skeleton”). The skeleton is a natural-born feature of a person, and can be described as a three-dimensional feature unique to the person.

### RELATED-ART DOCUMENT

### Patent Document

Patent Document 1: International Publication No. WO 2013/005447

### SUMMARY OF THE INVENTION

### Problems to be Solved by the Invention

However, so far, it has not been easy to measure a shape relating to a facial skeleton.

Hence, an object of the present invention is to obtain a shape relating to a facial skeleton easily.

### Means for Solving the Problems

A method according to an embodiment of the present invention includes a step of determining a nose feature of a user, and a step of estimating a shape relating to a facial skeleton of the user based on the nose feature of the user.

### Effects of the Invention

According to the present invention, it is possible to estimate a shape relating to a facial skeleton based on a nose feature.

### BRIEF DESCRIPTION OF THE DRAWINGS

FIG. 1 is a view illustrating an overall configuration according to an embodiment of the present invention.

FIG. 2 is a view illustrating functional blocks of a skeleton estimating device according to an embodiment of the present invention.

FIG. 3 is a flowchart illustrating a flow of a skeleton estimation process according to an embodiment of the present invention.

FIG. 4 is a view illustrating nose features according to an embodiment of the present invention.

FIG. 5 is a view illustrating extraction of a nose region according to an embodiment of the present invention.

FIG. 6 is a view illustrating calculation of nose feature quantities according to an embodiment of the present invention.

FIG. 7 illustrates an example of nose features of each face type according to an embodiment of the present invention.

FIG. 8 illustrates examples of faces estimated based on nose features according to an embodiment of the present invention.

FIG. 9 is a view illustrating a hardware configuration of a skeleton estimating device according to an embodiment of the present invention.

### DETAILED DESCRIPTION OF THE INVENTION

Each embodiment will be described below with reference to the attached drawings. In the specification and drawings, overlapping descriptions about components having substantially the same function and configuration will be omitted by denoting them by the same reference numerals.

### <Explanation of Terms>

“A shape relating to a facial skeleton” refers to either or both of a shape of a facial skeleton itself, and a shape of a face attributable to the skeleton. In the present invention, a shape relating to a facial skeleton is estimated from a nose feature based on correlation between the nose feature and the shape relating to the facial skeleton.

### <Overall Configuration>

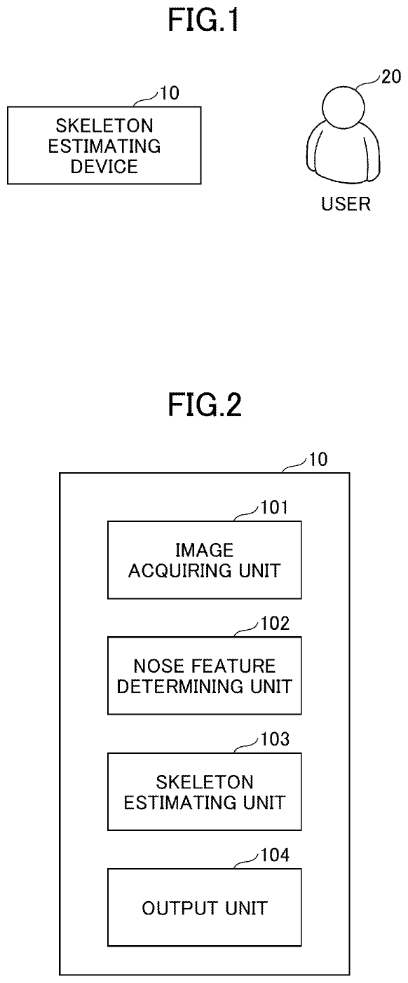

FIG. 1 is a view illustrating an overall configuration according to an embodiment of the present invention. A skeleton estimating device 10 is configured to estimate a shape relating to a facial skeleton of a user 20 based on a nose feature of the user 20. For example, the skeleton estimating device 10 is a smartphone including a camera function. The skeleton estimating device 10 will be described in detail below with reference to FIG. 2.

In the present specification, a case where the skeleton estimating device 10 is one device (e.g., a smartphone including a camera function) will be described. However, the skeleton estimating device 10 may be formed of a plurality of devices (e.g., a device free of a camera function and a digital camera). The camera function may be a function for capturing images of skin three-dimensionally, or may be a function for capturing images of skin two-dimensionally. Moreover any other device (e.g., a server) than the skeleton estimating device 10 may perform some of the processes described in the present specification as being performed by the skeleton estimating device 10.

### <Functional Blocks of Skeleton Estimating Device 10>

FIG. 2 is a view illustrating the functional blocks of the skeleton estimating device 10 according to an embodiment of the present invention. The skeleton estimating device 10 may include an image acquiring unit 101, a nose feature determining unit 102, a skeleton estimating unit 103, and an output unit 104. The skeleton estimating device 10 can function as the image acquiring unit 101, the nose feature determining unit 102, the skeleton estimating unit 103, and the output unit 104 by executing programs. Each will be described below.

The image acquiring unit 101 is configured to acquire an image including the nose of the user 20. An image including a nose may be an image in which a nose and parts other than the nose are captured (e.g., an image in which an entire face is captured), or an image in which only a nose is captured (e.g., an image in which the nose region of the user 20 is captured while being confined within a predetermined region displayed on a display device of the skeleton estimating device 10). When a nose feature is determined based on sources other than an image, the image acquiring unit 101 is not necessary.

The nose feature determining unit 102 is configured to determine a nose feature of the user 20. For example, the nose feature determining unit 102 determines a nose feature of the user 20 based on image information of the image (e.g., pixel values of the image) including the nose of the user 20 acquired by the image acquiring unit 101.

The skeleton estimating unit 103 is configured to estimate the shape relating to the facial skeleton of the user 20 based on the nose feature of the user 20 determined by the nose feature determining unit 102. For example, the skeleton estimating unit 103 sorts the shape relating to the facial skeleton of the user 20 based on the nose feature of the user 20 determined by the nose feature determining unit 102.

The output unit 104 is configured to output (e.g., display) information regarding the shape relating to the facial skeleton of the user 20 estimated by the skeleton estimating unit 103.

### <Nose Feature>

Here, a nose feature will be described. For example, a nose feature is at least one selected from a nasal root, a nasal bridge, a nasal apex, and nasal wings.

### <<Nasal Root>>

The nasal root is a base part of a nose. For example, a nose feature is at least one selected from whether the nasal root is high or low, the width of the nasal root, and a nasal root changing position at which the nasal root changes to become higher.

### <<Nasal Bridge>>

The nasal bridge is a part between the glabella and the nose tip. For example, a nose feature is at least one selected from whether the nasal bridge is high or low, and the width of the nasal bridge.

### <<Nasal Apex>>

The nasal apex is the most prominent part (nose tip) of the nose. For example, a nose feature is at least one selected from the roundness or sharpness of the nasal apex, and the direction of the nasal apex.

### <<Nasal Wings>>

The nasal wings are the projecting parts on both sides of the apex of the nose. For example, a nose feature is at least one selected from the roundness or sharpness of the nasal wings, and the size of the nasal wings.

### <Shape Relating to Facial Skeleton>

Here, the shape relating to the facial skeleton will be described. For example, the shape relating to the facial skeleton refers to, for example, the shape features of bones and the positional relationship and angles of the skeleton at at least one of an eye socket, a cheekbone, a nasal bone, a piriform aperture (a nasal cavity aperture opening to the face), a cephalic index, a maxilla bone, a mandible bone, a lip, a corner of a mouth, an eye, an epicanthic fold (an upper eyelid's skin fold that covers the inner corner of an eye), a facial contour, and positional relationship between an eye and an eyebrow (e.g., whether an eye and an eyebrow are apart or close). Examples of the shape relating to the facial skeleton will be presented below. The parenthesized contents represent specific examples of the items that are to be estimated.
Eye socket (laterally long, square shape, round)
Cheekbone, cheek (peak position, roundness)
Nasal bone (width, shape)
Piriform aperture (shape)
Cephalic index (width/depth of a skull bone=70, 75, 80, 85, 90)
Maxilla bone, maxilla (positional relationship with respect to an eye socket, nasolabial angle)
Mandible bone, mandible (depth size, depth angle, front angle, and contour shape (gill))
Forehead (forehead roundness, forehead shape)
Eyebrow (distance between an eye and an eyebrow, eyebrow shape, and eyebrow thickness)
Lip (the upper and lower lips are both thick, the lower lip is thick, the upper and lower lips are both thin, the lips are laterally long or short)
Corner of mouth (upcurved, downcurved, standard)
Eye (area, angle, distance between an eyebrow and an eye, and distance between the eyes)
Epicanthic fold (present, absent)
Facial contour (Rectangle, Round, Obal, Heart, Square, Average, Natural, Long)

### <Correspondence Relationship Between Nose Feature and Shape Relating to Facial Skeleton>

Here, the correspondence relationship between a nose feature and a shape relating to a facial skeleton will be described. In the present invention, a shape relating to a facial skeleton is estimated based on the correspondence relationship between nose features and shapes relating to facial skeletons, the correspondence relationship being previously stored in, for example, the skeleton estimating device 10. A shape relating to a facial skeleton may be estimated based not only on a nose feature, but also on a part of a nose feature and of a facial feature.

The correspondence relationship may be a database that is previously designated, or may be a trained model obtained by machine learning. In a database, nose features (may also be parts of nose features and of facial features) and shapes relating to facial skeletons are associated with each other. A trained model is a forecasting model configured to output information regarding a shape relating to a facial skeleton in response to an input of information regarding a nose feature (may also be a part of a nose feature and of a facial feature). The correspondence relationship between nose features and shapes relating to facial skeletons may be generated per group sorted based on factors that may affect the skeleton (e.g., Caucasoid, Mongoloid, Negroid, and Australoid).

### <<Generation of Trained Model>>

In an embodiment of the present invention, a computer such as the skeleton estimating device 10 can generate a trained model. Specifically, a computer such as the skeleton estimating device 10 can acquire training data including input data representing nose features (may be parts of nose features and of facial features) and output data representing shapes relating to facial skeletons, perform machine learning using the training data, and generate a trained model configured to output a shape relating to a facial skeleton in response to an input of a nose feature (may also be a part of a nose feature and of a facial feature). Through machine learning using the training data including input data representing nose features (may also be parts of nose features and of facial features) and output data representing shapes relating to facial skeletons, a trained model configured to output a shape relating to a facial skeleton in response to an input of a nose feature (may also be a part of a nose feature and of a facial feature) is generated.

Examples of estimation based on the correspondence relationship between nose features and shapes relating to facial skeletons will be described below.

### Estimation Example 1

For example, the skeleton estimating unit 103 can estimate the cephalic index based on whether the nasal root is high or low, the nasal root height changing position, and whether the nasal bridge is high or low. Specifically, the skeleton estimating unit 103 estimates the cephalic index to be lower, the higher either or both of the nasal root and the nasal bridge are.

### Estimation Example 2

For example, the skeleton estimating unit 103 can estimate whether the corners of the mouth are upcurved or downcurved based on the width of the nasal bridge. Specifically, the skeleton estimating unit 103 estimates the corners of the mouth to be more downcurved, the greater the width of the nasal bridge is.

### Estimation Example 3

For example, the skeleton estimating unit 103 can estimate the size and thickness of the lips (1. the upper and lower lips are both long and thick, 2. the lower lip is thick, and 3. the upper and lower lips are both thin and short) based on the roundness of the nasal wings and the sharpness of the nasal apex.

### Estimation Example 4

For example, the skeleton estimating unit 103 can estimate presence or absence of the epicanthic folds based on the nasal root. Specifically, the skeleton estimating unit 103 estimates that the epicanthic folds are present when it is determined that the nasal root is low.

### Estimation Example 5

For example, the skeleton estimating unit 103 can sort the shape of the mandible (into, for example, three categories) based on whether the nasal bridge is low or high, the height of the nasal root, and the roundness and size of the nasal wings.

### Estimation Example 6

For example, the skeleton estimating unit 103 can estimate the piriform aperture based on the height of the nasal bridge.

### Estimation Example 7

For example, the skeleton estimating unit 103 can estimate the distance between the eyes based on how low the nasal bridge is. Specifically, the skeleton estimating unit 103 estimates the distance between the eyes to be greater, the lower the nasal bridge is.

### Estimation Example 8

For example, the skeleton estimating unit 103 can estimate the roundness of the forehead based on the height of the nasal root and the height of the nasal bridge.

### Estimation Example 9

For example, the skeleton estimating unit 103 can estimate the distance between an eye and an eyebrow and the shape of the eyebrow based on whether the nasal bridge is high or low, the size of the nasal wings, and the nasal root height changing position.

### <Processing Method>

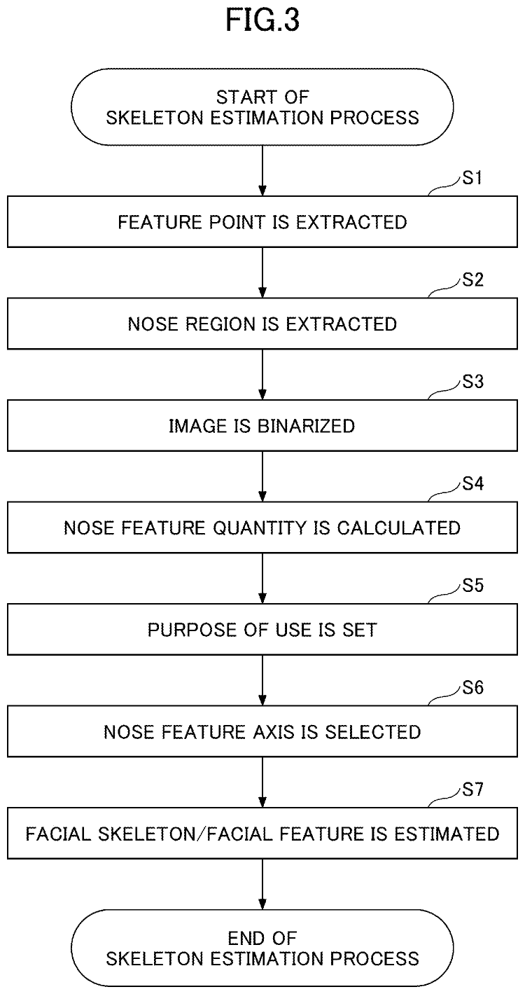

FIG. 3 is a flowchart illustrating the flow of the skeleton estimation process according to an embodiment of the present invention.

In the step 1 (S1), the nose feature determining unit 102 extracts feature points (e.g., feature points at an inner end of an eyebrow, an inner corner of an eye, and the nose tip) from an image including a nose.

In the step 2 (S2), the nose feature determining unit 102 extracts a nose region based on the feature points extracted in S1.

When the image including the nose is an image in which only the nose is captured (e.g., an image in which the nose region of the user 20 is captured while being confined within a predetermined region displayed on a display device of the skeleton estimating device 10), the image in which only the nose is captured is used as is (i.e., S1 may be omitted).

In the step 3 (S3), the nose feature determining unit 102 reduces the number of gradation levels in the image representing the nose region extracted in S2 (e.g., binarizes the image). For example, the nose feature determining unit 102 reduces the number of gradation levels in the image representing the nose region, by using at least one selected from brightness, luminance, Blue of RGB, and Green of RGB. S3 may optionally be omitted.

In the step 4 (S4), the nose feature determining unit 102 calculates nose feature quantities based on image information of the image (e.g., pixel values of the image) representing the nose region. For example, the nose feature determining unit 102 calculates the average of the pixel values in the nose region, the number of pixels that are lower than or equal to, or higher than or equal to a predetermined value, cumulative pixel values, and a pixel value changing quantity as the nose feature quantities.

In the step 5 (S5), the skeleton estimating unit 103 sets a purpose of use (i.e., for what the information regarding a shape relating to a facial skeleton is used (e.g., proposals for, for example, skeletal diagnosis, use of beauty equipment, makeup, hair style, and eyeglasses)). For example, the skeleton estimating unit 103 sets a purpose of use in accordance with an instruction from the user 20. S5 may optionally be omitted.

In the step 6 (S6), the skeleton estimating unit 103 selects a nose feature axis based on the purpose of use set in S5. The nose feature axis indicates one or a plurality of nose features that is or are used for the purpose of use set in S5 (i.e., is or are used for estimating a shape relating to a facial skeleton).

In the step 7 (S7), the skeleton estimating unit 103 estimates a shape relating to a facial skeleton. Specifically, the skeleton estimating unit 103 determines one or a plurality of nose features that is or are indicated by the nose feature axis selected in S6 based on the nose feature quantities calculated in S4. Next, the skeleton estimating unit 103 estimates a shape relating to a facial skeleton based on the determined nose feature(s).

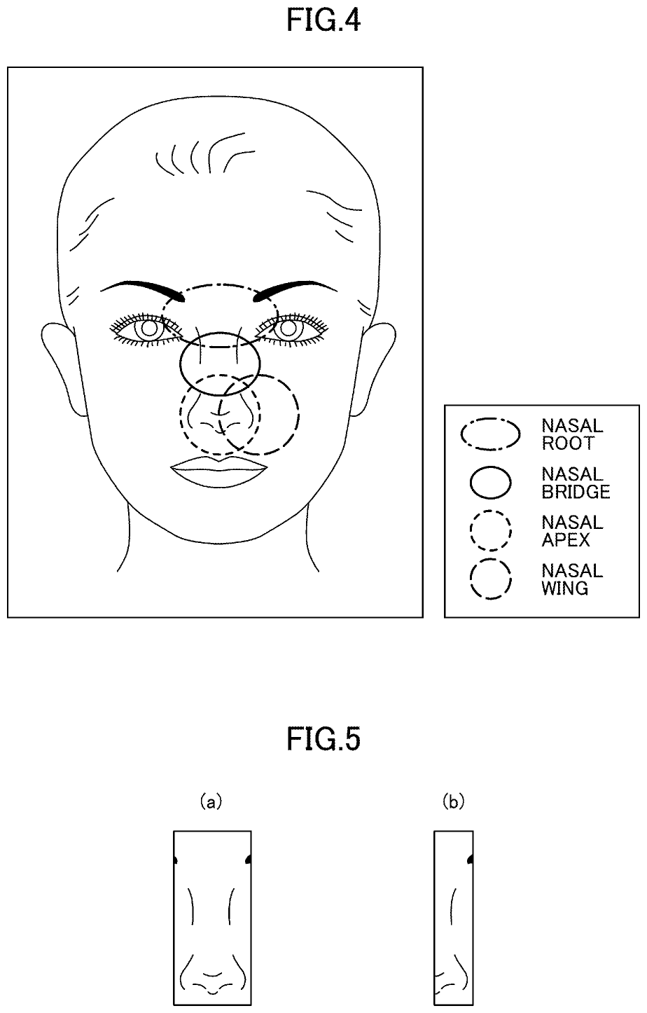

FIG. 4 is a view illustrating nose features according to an embodiment of the present invention. As described above, a nose feature is at least one selected from a nasal root, a nasal bridge, a nasal apex, and nasal wings. FIG. 4 illustrates the positions of a nasal root, a nasal bridge, a nasal apex, and nasal wings.

### <Extraction of Nose Region>

FIG. 5 is a view illustrating extraction of a nose region according to an embodiment of the present invention. The nose feature determining unit 102 extracts a nose region of an image including a nose. For example, a nose region may be the entirety of a nose as in FIG. 5 (a), or may be a part of a nose (e.g., a right half or a left half) as in FIG. 5 (b).

### <Calculation of Nose Feature Quantities>

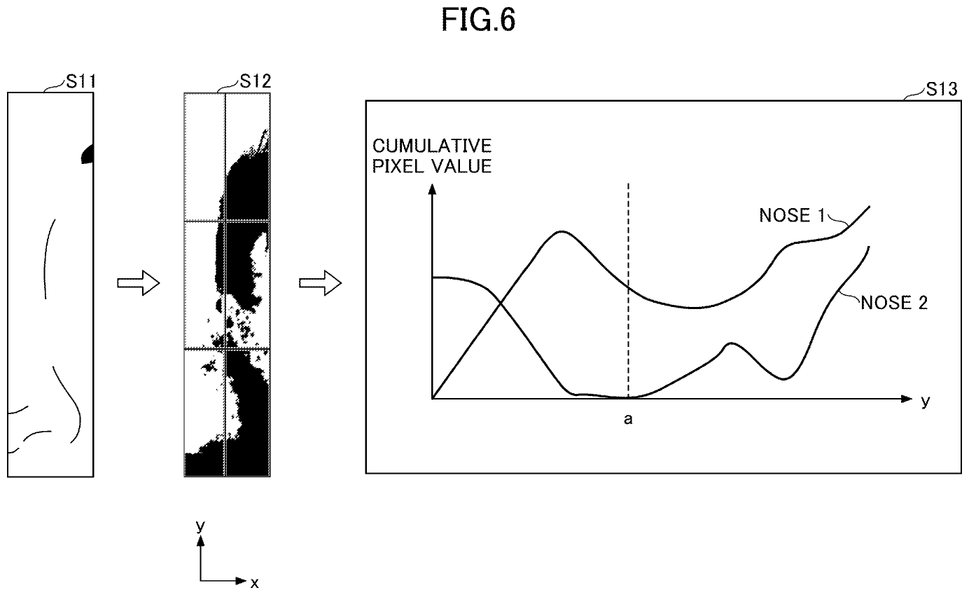

FIG. 6 is a view illustrating calculation of nose feature quantities according to an embodiment of the present invention.

In the step 11 (S11), a nose region in an image including a nose is extracted.

In the step 12 (S12), the number of gradation levels in the image representing the nose region extracted in S11 is reduced (e.g., the image is binarized). S12 may optionally be omitted.

In the step 13 (S13), nose feature quantities are calculated. FIG. 6 represents cumulative pixel values by setting the high brightness side at 0 and the low brightness side at 255. For example, the nose feature determining unit 102 normalizes a plurality of regions (e.g., separate regions illustrated in S12) one by one. Next, the nose feature determining unit 102 calculates, as the nose feature quantities, for example, the average pixel value, the number of pixels that are lower than or equal to, or higher than or equal to a predetermined value, a cumulative pixel value in either or both of the X direction and the Y direction, and a pixel value changing quantity in either or both of the X direction and the Y direction, region by region (e.g., by using data of the image at a lower brightness side or a higher brightness side). In S13 of FIG. 6, a cumulative pixel value in the X direction is calculated for each Y-direction position.

The method for calculating each feature quantity will be described below.

For example, the feature quantity of the nasal root is the feature quantity of an upper region (close to an eye) among the separate regions illustrated in S12. The feature quantity of the nasal bridge is the feature quantity of an upper or a central region among the separate regions illustrated in S12. The feature quantities of the nasal apex and the nasal wings are the feature quantities of lower regions (close to the mouth) among the separate regions illustrated in S12. These nose feature quantities are normalized by the distance between the eyes.
Height of a nasal root: Whether the nasal root is high or low is determined based on the pixel value changing quantity in the Y direction in an upper region of the nose. The height of the nasal root may be calculated as a value indicating whether the nasal root is high or low, or the nasal root may be sorted as being high or low. It can be seen from S13 that the nasal root height changing position of the nose 2 is located at an upper region, since the value of the nose 2 changes instantly in the Y direction.
Width of a nasal root: The width of the nasal root is determined based on a pattern of average pixel values in a plurality of (e.g., from 2 to 4) regions, into which an upper region of the nose is divided in the X direction.
Height of a nasal bridge: Whether the nasal bridge is high or low is determined based on the average cumulative pixel value in the central region of the nose. The height of the nasal bridge may be calculated as a value indicating whether the nasal bridge is high or low, or the nasal bridge may be sorted as being high or low.
Width of a nasal bridge: The width of the nasal bridge is determined based on a pattern of average pixel values in a plurality of (e.g., from 2 to 4) regions, into which the central region of the nose is divided in the X direction.
Roundness or sharpness of a nasal apex: Roundness or sharpness of the nasal apex is determined based on other nose features (height of nasal bridge, and roundness or sharpness of nasal wings). The lower the nasal bridge is and the rounder the nasal wings are, the rounder the nasal apex is.
Direction of a nasal apex: The direction of the nasal apex is determined based on a width from the downmost position of the nose to a position that is at a predetermined percentage of the maximum X-direction cumulative pixel value in the central region of the nose. The greater this width is, the more upturned the nasal apex is.
Roundness or sharpness of nasal wings: The roundness or sharpness of the nasal wings is determined based on the value changing quantity in the Y direction in a lower region of the nose.
Size of nasal wings: The size of the nasal wings is determined based on the number percentage of pixels that are lower than or equal to a predetermined value in the central portion of a lower region. The greater the number of such pixels is, the larger the nasal wings are.

### <<Face Type>>

As described above, “a shape relating to a facial skeleton” refers to either or both of “the shape of the facial skeleton itself” and “the shape of the face attributable to the skeleton”. “A shape relating to a facial skeleton” can encompass face type.

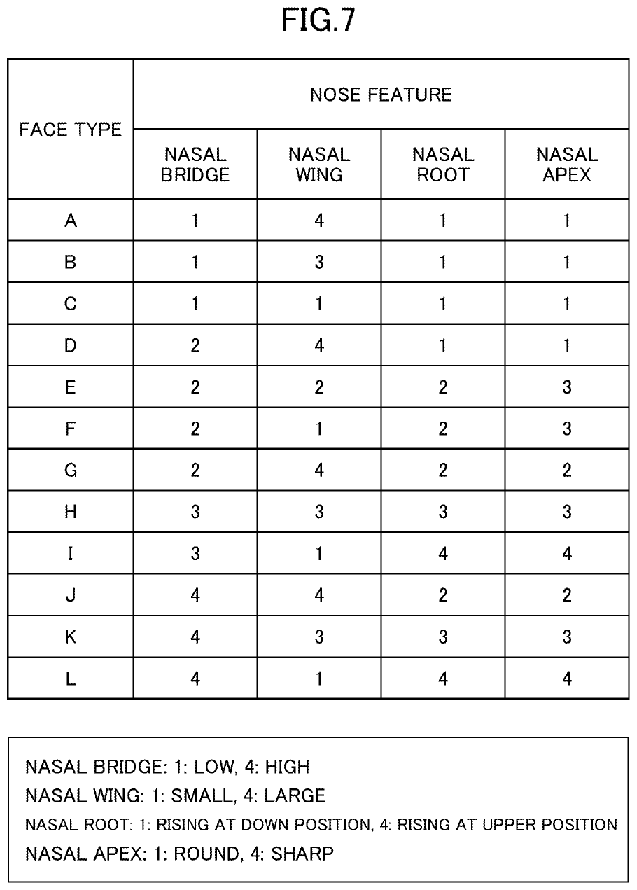

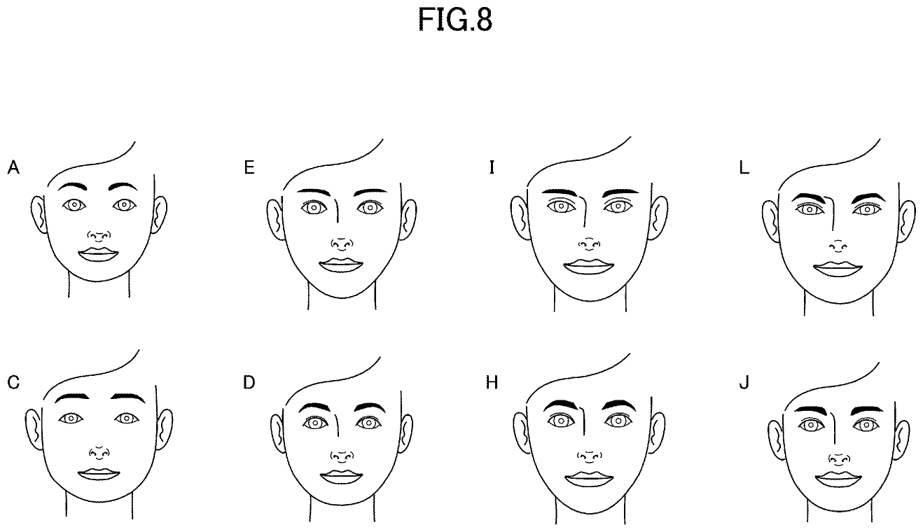

In an embodiment of the present invention, it is possible to estimate which face type of a plurality of face types (specifically, face types that are sorted based on either or both of “the shape of the facial skeleton itself” and “the shape of the face attributable to the skeleton”) the face of a user is, based on the nose features of the user. Face types will be described below with reference to FIG. 7 and FIG. 8.

FIG. 7 is an example of nose features of each face type according to an embodiment of the present invention. FIG. 7 indicates nose features of each face type (each of face types A to L). A face type may be estimated using all (four) of a nasal bridge, nasal wings, a nasal root, and a nasal apex, or a face type may be estimated using some of these features (e.g., two features, namely a nasal bridge and nasal wings, two features, namely a nasal bridge and a nasal root, only a nasal bridge, or only nasal wings).

In this way, a face type is estimated based on nose features. For example, it is estimated that the eyes are round, that the eyes are inclined downward, that the eye size is small, that the eyebrow shape is an arch-like, that the positions of the eyebrows and the eyes are apart, and that the facial contour is ROUND, from the nose features of the face type A. Moreover, for example, it is estimated that the eyes are sharp, that the eyes are inclined considerably upward, that the eye size is large, the eyebrow shape is sharp, that the positions of the eyebrows and the eyes are considerably close, and that the facial contour is RECTANGLE, from the nose features of the face type L.

FIG. 8 illustrates examples of faces estimated based on nose features according to an embodiment of the present invention. According to an embodiment of the present invention, it is possible to estimate which face type of various face types as illustrated in FIG. 8 the face of a user is, based on the nose features of the user.

Hence, it is possible to sort face types based on feature quantities of the nose that tends not to be affected by lifestyle habits or conditions during image capturing. For example, it is possible to utilize face types that are sorted based on nose features, when showing makeup guidance or skin characteristics (e.g., it is possible to show makeup guidance or skin characteristics based on what facial features a face type concerned has or what impression a face type concerned would give).

### <Effects>

Hence, according to the present invention, it is possible to estimate a shape relating to a facial skeleton (i.e., either or both of the shape of the facial skeleton itself and the shape of the face attributable to the skeleton) easily based on nose features, without an actual measurement. In an embodiment of the present invention, it is possible to propose, for example, skeletal diagnosis, and use of beauty equipment, makeup, hair style, and eyeglasses that are suited to a person concerned, based on a shape relating to a facial skeleton estimated based on nose features.

### <Hardware Configuration>

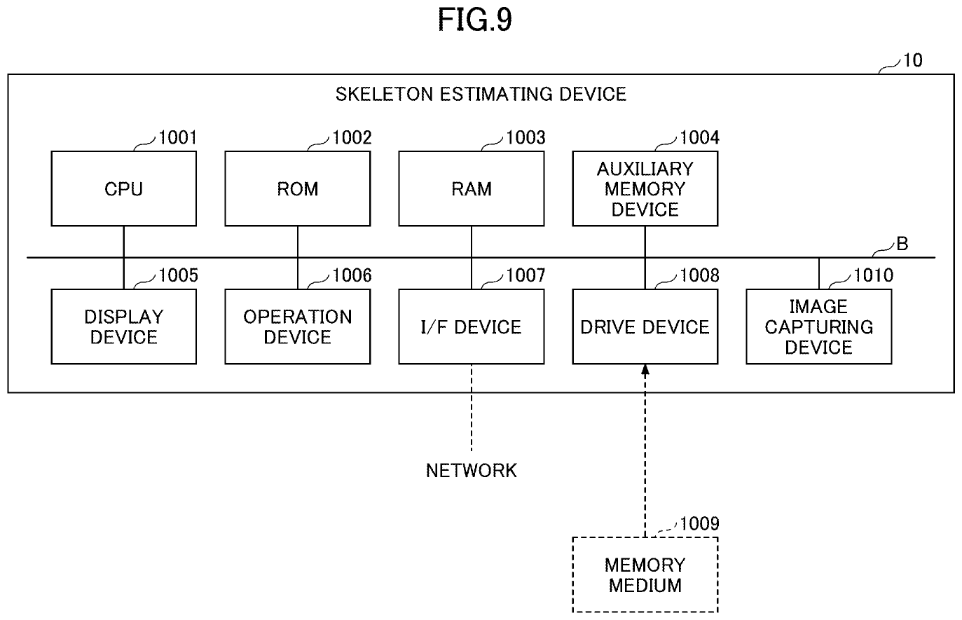

FIG. 9 is a view illustrating the hardware configuration of the skeleton estimating device 10 according to an embodiment of the present invention. The skeleton estimating device 10 includes a Central Processing Unit (CPU) 1001, a Read Only Memory (ROM) 1002, and a Random Access Memory (RAM) 1003. The CPU 1001, the ROM 1002, and the RAM 1003 form what is generally referred to as a computer.

The skeleton estimating device 10 may include an auxiliary memory device 1004, a display device 1005, an operation device 1006, an Interface (I/F) device 1007, and a drive device 1008.

The respective hardware pieces of the skeleton estimating device 10 are mutually coupled via a bus B.

The CPU 1001 is an operation device configured to execute various programs installed on the auxiliary memory device 1004.

The ROM 1002 is a nonvolatile memory. The ROM 1002 functions as a main memory device configured to store various programs and data that are necessary for the CPU 1001 to execute the various programs installed on the auxiliary memory device 1004. Specifically, the ROM 1002 functions as a main memory device configured to store, for example, boot programs such as Basic Input/Output System (BIOS) and Extensible Firmware Interface (EFI).

The RAM 1003 is a volatile memory such as a Dynamic Random Access Memory (DRAM) and a Static Random Access Memory (SRAM). The RAM 1003 functions as a main memory device that provides a work area in which the various programs installed on the auxiliary memory device 1004 are spread when executed by the CPU 1001.

The auxiliary memory device 1004 is an auxiliary memory device configured to store various programs and information used when the various programs are executed.

The display device 1005 is a display device configured to display, for example, the internal status of the skeleton estimating device 10.

The operation device 1006 is an input device by which an operator of the skeleton estimating device 10 inputs various instructions into the skeleton estimating device 10.

The I/F device 1007 is a communication device configured to connect to a network in order to communicate with other devices.

The drive device 1008 is a device configured for a memory medium 1009 to be set therein. The memory medium 1009 meant here encompasses media configured to record information optically, electrically, or magnetically, such as a CD-ROM, a flexible disk, and a magneto-optical disk. The memory medium 1009 may also encompass, for example, semiconductor memories configured to record information electrically, such as an Erasable Programmable Read Only Memory (EPROM) and a flash memory.

The various programs to be installed on the auxiliary memory device 1004 are installed by a distributed memory medium 1009 being set in the drive device 1008 and the various programs recorded in the memory medium 1009 being read out by the drive device 1008. Alternatively, the various programs to be installed on the auxiliary memory device 1004 may be installed by being downloaded from a network via the I/F device 1007.

The skeleton estimating device 10 includes an image capturing device 1010. The image capturing device 1010 is configured to capture an image of the user 20.

Examples of the present invention have been described in detail above. However, the present invention is not limited to the specific embodiments described above, and various modifications and changes may be applied to the present invention within the scope of the spirit of the present invention described in the claims.

The present international application claims priority to Japanese Patent Application No. 2021-021915 filed Feb. 15, 2021, and the entire contents of Japanese Patent Application No. 2021-021915 are incorporated herein by reference.

### DESCRIPTION OF THE REFERENCE NUMERALS

10: skeleton estimating device
20: user
101: image acquiring unit
102: nose feature determining unit
103: skeleton estimating unit
104: output unit
1001: CPU
1002: ROM
1003: RAM
1004: auxiliary memory device
1005: display device
1006: operation device
1007: I/F device
1008: drive device
1009: memory medium
1010: image capturing device

1. A skeleton estimating method, comprising:
determining a nose feature of a user; and
estimating a shape relating to a facial skeleton of the user based on the nose feature of the user.

2. The skeleton estimating method according to claim 1, further comprising:
acquiring an image including a nose of the user,
wherein the nose feature of the user is determined based on image information of the image.

3. The skeleton estimating method according to claim 1,
wherein the estimating includes sorting the shape relating to the facial skeleton of the user.

4. The skeleton estimating method according to claim 1,
wherein the estimating includes estimating which of face types a face of the user is, the face types being sorted based on a shape relating to a facial skeleton.

5. The skeleton estimating method according to claim 1,
wherein the shape relating to the facial skeleton of the user is either or both of a shape of the facial skeleton of the user, and a shape of a face of the user attributable to the facial skeleton of the user.

6. The skeleton estimating method according to claim 1,
wherein the nose feature is at least one selected from a nasal root, a nasal bridge, a nasal apex, and nasal wings.

7. The skeleton estimating method according to claim 1,
wherein the shape relating to the facial skeleton of the user is estimated using a trained model configured to output the shape relating to the facial skeleton in response to an input of the nose feature.

8. A skeleton estimating device, comprising:
a nose feature determining unit configured to determine a nose feature of a user; and
a skeleton estimating unit configured to estimate a shape relating to a facial skeleton of the user based on the nose feature of the user.

9. A non-transitory computer-readable recording medium storing a program causing a computer to function as:
a nose feature determining unit configured to determine a nose feature of a user; and
a skeleton estimating unit configured to estimate a shape relating to a facial skeleton of the user based on the nose feature of the user.

10. A system including a skeleton estimating device and a server, the system comprising:
a nose feature determining unit configured to determine a nose feature of a user; and
a skeleton estimating unit configured to estimate a shape relating to a facial skeleton of the user based on the nose feature of the user.

11. A trained model generating method, comprising:
acquiring training data including input data representing a nose feature and output data representing a shape relating to a facial skeleton; and
performing machine learning using the training data, to generate a trained model configured to output the shape relating to the facial skeleton in response to an input of the nose feature.

12. A trained model generated by machine learning using training data including input data representing a nose feature and output data representing a shape relating to a facial skeleton, the trained model being configured to output the shape relating to the facial skeleton in response to an input of the nose feature.
